(19) United States
(12) Patent Application Publication
(10) Pub. No.: US 2017/0112787 A1
(43) Pub. Date: Apr. 27, 2017

(54) METHODS OF TREATMENT OF INFLAMMATION OF THE GUT

(71) Applicant: THE UNIVERSITY OF CONNECTICUT, Farmington, CT (US)

(72) Inventors: PRAMOD SRIVASTAVA, AVON, CT (US); NANDINI ACHARYA, ALLSTON, MA (US); SREYASHI BASU, HOUSTON, TX (US)

(21) Appl. No.: 15/147,022

(22) Filed: May 5, 2016

Related U.S. Application Data
(60) Provisional application No. 62/158,730, filed on May 8, 2015.

(51) Int. Cl. A61K 31/16; A61K 45/06; A61K 9/00

(52) U.S. Cl. CPC A61K 31/16; A61K 9/0053; A61K 45/06

(57) ABSTRACT

Described herein are methods of improving immune homeostasis in the gut of a subject suffering from an autoimmune disease characterized by inflammation of the gut by administering, e.g., orally administering, to the subject an effective amount of a cannabinoid receptor agonist to improve immune homeostasis in the gut of the subject. Exemplary cannabinoid receptor ligands include Anandamide. The methods are particularly useful to treat gut inflammation associated with diabetes mellitus Type I.

### CROSS-REFERENCE TO RELATED APPLICATIONS

This application claims priority to U.S. Provisional Application 62/158,730 filed on May 8, 2015, which is incorporated herein by reference in its entirety.

### STATEMENT REGARDING FEDERALLY SPONSORED RESEARCH & DEVELOPMENT

This invention was made with government support under grant AI095776 awarded by the National Institutes of Health. The government has certain rights in the invention.

### FIELD OF THE DISCLOSURE

The present disclosure is related to new uses of cannabinoid receptor ligands such as the endocannabinoids.

### BACKGROUND

In autoimmune disease, the immune system attacks the body's own tissues. In many autoimmune diseases, the immune system attacks part of the gastrointestinal tract resulting in chronic gut inflammation. Autoimmune diseases with gastrointestinal inflammation include celiac disease, Crohn's disease, ulcerative colitis, autoimmune hepatitis, inflammatory bowel disease, and primary biliary cirrhosis. Even systemic autoimmune disorders such as lupus can result in gastrointestinal inflammation. While the treatment of autoimmune disease typically depends on the disease, a major goal of treatment is the reduction of inflammation. Thus, treatments that reduce gut inflammation would be helpful in treating a wide variety of autoimmune disorders.

What is needed are therapies useful to reduce gut inflammation and promote gut homeostasis in subjects with autoimmune diseases.

### BRIEF SUMMARY

In one aspect, a method of improving immune homeostasis in the gut of a subject suffering from an autoimmune disease characterized by inflammation of the gut comprises administering to the subject an effective amount of a cannabinoid receptor ligand to improve immune homeostasis in the gut of the subject, wherein the gut includes the gastrointestinal tract as well as organs served by the blood supply to the gut.

In another aspect, a method of improving the symptoms of gut inflammation associated with diabetes mellitus Type I in a subject suffering from diabetes mellitus Type I comprises administering to the subject an effective amount of a cannabinoid receptor ligand to reduce the symptoms of gut inflammation in the subject.

### BRIEF DESCRIPTION OF THE DRAWINGS

FIGS. 1A-C show that CX3CR1^{hi} Mφ express high levels of VR1 and CB2. FIG. 1(A) Gating strategy for small intestinal Lamina Propria (siLP) cells. siLP cells were purified from CX3CR1^{gfp/+} mice and analyzed using flow cytometry. SSC^{hi} and SSC^{lo} cells were gated. CD11b⁺SiglecF⁺ cells were analyzed in the SSC^{hi} population. SSC^{lo} cells were gated for CD3⁺ and B220⁺cells and mononuclear-phagocytes comprising of R1 (CD11b⁺CD11c⁺) and R2 (CD11b⁻CD11c⁺) cells. R1 and R2 were further analyzed for expression of CX3CR1 and/or CD103. Selected gates are boxed whose VR1 and CB2 expression levels are shown in (B) and (C) respectively. FIG. 1B and FIG. 1C are histograms of VR1 and CB2 expression in selected populations from (A). Histograms with the isotype control antibody are shown in gray and the histograms of expression of VR1 or CB2 by the selected population from (A) are shown to the right of the control. The number inside each box represent the geometric mean fluorescence intensity (GMFI), the top numbers represent the GMFI of the isotype control antibody and the bottom numbers represent GMFI of anti-VR1 or anti-CB2 antibody for the given population.

FIG. 2A-G show the endocannabinoid system influences the siLP CX3CR1hi Mφ. FIG. 2(A) Geometric mean fluorescence intensity (GMFI) of VR1 and CB2 on indicated siLP cells (* and ° represent statistical comparison of VR1 and CB2 expression respectively, of each indicated cell type with expression of these receptors by CD11b+CX3CR1hi Mφ). FIG. 2(B) Phenotype of siLP CD11b+CX3CR1hi cells (top panel); expression of IL10 by CD11b+CX3CR1hi Mφ (Q1), CD11b+CX3CR1lo and CD11b+CX3CR1-cells (Q2) (bottom panel). FIG. 2(C) Genotype of the mice constructed (left panel). Frequency of CD11b+CX3CR1hi Mφ and CD11b+CX3CR1lo cells in the siLP of CX3CR1gfp/+ VR1+/+ and CX3CR1gfp/+ VR1-/-mice (middle panel). Absolute numbers of CD11b+ CX3CR1hi Mφ and CD11b+CX3CR1lo cells in the siLP (n=3) (right panel). FIG. 2(D) CP elicited changes (24 hours after feeding) in the frequency (top and bottom left panel) and absolute numbers (top and bottom right panel) of CX3CR1hi Mφ and CX3CR1lo cells in the siLP. Changes in the frequency (24 hours after feeding) of siLP CX3CR1hi Mφ elicited by FIG. 2(E) AEA and FIG. 2(F) PF3845 (n=4-5). FIG. 2(G) CP elicited changes in siLP CX3CR1hi Mφ of NOD mice (n=3). (ns, not significant; *p<0.05; **p<0.01; ***p<0.001; °°p<0.01; unpaired Student's t test, data represent mean±SEM).

FIG. 3 shows expression of macrophage marker CD64 by siLP CX3CR1^{lo} cells CD103⁺DCs. Contour plot shows the expression of CD64 and CD103 on CD11b⁺CX3CR1^{lo} cells (left panel) and CD11c⁺CD103⁺DCs (right panel).

FIG. 4 shows CX3CR1-antibody staining correlates with CX3CR1-GFP expression in CX3CR1^{gfp/+} mice. Dot plot represents 3 different populations shown by gates in distinct colors: CD11b⁺CX3CR1^{hi} Mφ (top box, right histogram), CD11b⁺CX3CR1^{lo} (middle box, left histogram) and CD11b⁺CX3CR1⁻ cells (bottom box, middle histogram) among live SSC^{lo} siLP cells of CX3CR1^{gfp/+} mice gated based on staining with CX3CR1 antibody (left panel). Histograms represent expression of CX3CR1-GFP by each of the gated populations (right panel), showing the correlation between CX3CR1-antibody staining and CX3CR1-GFP among live SSC^{lo} CD11b⁺ siLP cells of CX3CR1^{gfp/+}.

FIG. 5 shows that VR1 regulates siLP CX3CR1^{hi} Mφ. Bar graph represents the frequency of CX3CR1^{hi} Mφ among live SSC^{lo} siLP cells were analyzed by flow cytometry in VR1^{+/+} and VR1^{-/-} mice using antibody staining for CX3CR1 (n=3, *p<0.05, unpaired Student's t test, data represent mean±SEM).

FIGS. 6A and B shows that VR1 does not affect the proportion and total numbers of DCs and lymphocytes in siLP. FIG. 6(A-B) siLP cells were purified from CX3CR1^{gfp/+} VR1^{+/+} and CX3CR1^{gfp/+} VR^{-/-} mice and were analyzed by flow cytometry. FIG. 6(A) Column scatter graphs representing Frequency of CD 103⁺ DCs, CD3⁺ and B220⁺ cells among live SSC^{lo} siLP cells in the VR1^{+/+} and VR1^{-/-} mice. FIG. 6(B) Bar graphs showing the absolute numbers of CD11c⁺CD103⁺ DCs, CD3⁺ and B220⁺ cells in the CX3CR1^{gfp/+} VR1^{+/+} and CX3CR1^{gfp/+} VR1^{-/-} mice. (ns, not significant, data represent mean±SEM).

FIG. 7 shows VR1 mediated effects on CX3CR1^{hi} Mφ requires the presence of functional CX3CR1. CX3CR1^{gfp/gfp} mice were orally gavaged with Vehicle or Capsaicin (CP) (10 μg). siLP cells were isolated after 24 h and analyzed by flow cytometry. Bar graph represents CP elicited changes in the siLP CX3CR1 Mφ among live SSC^{lo} siLP. (ns, not significant, data represent mean±SEM).

FIG. 8A-D show the engagement of VR1 enhances the tolerogenic properties of siLP MNPs. FIG. 8(A) CP elicited changes in GMFI of IL10 and TNFα in CD11b+CX3CR1hi Mφ (n=4). FIG. 8(B) CP elicited changes in GMFI of CX3CR1 by CD11b+ CX3CR1hi Mφ (n=4). (C-D) Mice were orally gavaged with Vehicle or CP (10 μg) and siLP MNPs (CD11b+CD11c+ and CD11c+) were sorted after 24 hours (C) and 48 hours (D). FIG. 8(C) Sorted MNPs were co-cultured (1:1) with naïve splenic CD4+CD25−CD62L+ T cells derived from either IL27RA+/+ or IL27RA−/−mice for 4 days. Expression of IL10 and IFN-γ0 among the CD4+ cells were analyzed by flow cytometry. Bar graphs represent the frequency of CD4+IL10+, CD4+IFN-y+ and CD4+IL10+IFN-y+ (Tr1). Data are representative of two independent experiments and show mean values±SEM of duplicate or triplicate determinations where MNPs were sorted from 12 pooled mice per group. FIG. 8(D) Total RNA was extracted from sorted MNPs and expression levels of IL27-p28 were evaluated by qPCR. Data represent duplicate determinations from 12 pooled mice. Bar graph represents fold increase in expression of IL27-p28 in CP treated samples with respect to vehicle treated samples. (ns, not significant *p<0.05, **p<0.01, ***p<0.001; unpaired Student's t test, data represent mean±SEM).

FIG. 9 shows CP increases IL10 expression in CD103⁺DCs. IL10-GFP reporter mice were orally gavaged with Vehicle or Capsaicin (CP) (10 μg). siLP cells were isolated after 24 hrs and CP elicited changes in IL10 expression among siLP CD11c⁺CD103⁺ DCs were analyzed by flow cytometry. Column scatter graphs represents GMFI of IL10 among siLP CD11c⁺CD103⁺ DCs. (n=3, *p<0.05, unpaired Student's t test, data represent mean±SEM).

FIG. 10 shows the gating strategy for sorting siLP MNPs. siLP cells were harvested and SSC^{lo}, live were sorted on the basis of the expression of CD11b and CD11c after exclusion of doublets.

FIG. 11A-D shows VR1 plays a critical role in the homeostasis of CX3CR1hi Mφ in the PLN but not in the MLN. FIG. 11(A) Frequency of CX3CR1+ cells among live FSChi PLN cells (left panel) and MLN cells (right panel) in VR1+/+ and VR1−/− mice. FIG. 11(B) Frequency of CD11b+CX3CR1+ Mφ among live FSChi PLN cells (left panel) and live FSChi MLN cells (right panel) 3 days post oral gavage with CP (10 μg). FIG. 11(C) Frequency of PLN CD11b+CX3CR1+ Mφ of NOD mice 3 days post oral gavage with CP (10 μg). FIG. 11(D) Dot plot shows the gate for PLN CD11b+CX3CR1+ cells (red gate). Contour plot shows the expression of MHCII and CD11c on the gated cells. Left histogram shows the expression of CD64 (no fill) and isotype control (shaded) by the gated cells; right histogram shows IL10 expression by gated cells from IL10-GFP reporter mouse (no fill) and C57BL/6 mouse with no GFP (shaded). (For (A) and (C) data were pooled from two independent experiments totaling seven to nine mice per group. ns, not significant, **p<0.01, ***p<0.001, ****p<0.0001; unpaired Student's t test).

FIG. 12A-G show engagement of VR1 expands a tolerogenic subset of CD4+T cells in an IL27 dependent manner. FIG. 12(A-B) Frequency of CP elicited CD4+IL10+ IFN-γ+ (Tr1) cells among (A) live SSClo siLP cells (n=4) and (B) live PLN cells of NOD mice (n=4). FIG. 12(C to E) Frequency of CP elicited Tr1 cells among live PLN cells in (C) IL27RA+/+ and IL27RA−/− (n=4), (D) IL27RA+/+, IL27RA−/− and IL27RA+/− mice (n=3-4) and (E) CX3CR1gfp/gfp mice (n=4). FIG. 12(F) Frequency of CP elicited CD4+IL17A+ cells among live CD4+ PLN cells of NOD mice (n=4). FIG. 12(G) Schematic representation of the experimental design (top panel). Survival curve represents difference in diabetes development between the two recipient groups (bottom panel) (n=15). (Data are representative of two to five independent experiments, ns, not significant; *p<0.05; **p<0.01; ****p<0.0001; unpaired Student's t test, error bars indicate±SEM).

FIG. 13 further shows that oral administration of Anandamide (AEA) provides protection from Type 1 Diabetes (T1D). Female NOD mice were orally administered AEA or vehicle at 9^{th} and 10^{th} week of age and urine glucose was monitored to study T1D disease progression (n=10, *p<0.05, Mantel-Cox test).

The above-described and other features will be appreciated and understood by those skilled in the art from the following detailed description, drawings, and appended claims.

### DETAILED DESCRIPTION

Disclosed herein are methods of improving immune homeostasis in the gut of a subject suffering from an autoimmune disease characterized by inflammation of the gut by administering, e.g., orally administering, to the subject an effective amount of a cannabinoid receptor ligand to improve immune homeostasis in the gut. The Endocannabinoid Anandamide is an intestinal endocannabinoid which engages cannabinoid receptors (CB) on the enteric nervous system and controls appetite and energy balance. Anandamide can also engage VR1, a nonselective cation channel in neurons. The inventors of the present application have found that the Endocannabinoid Anandamide, a cannabinoid receptor ligand, can maintain immune homeostasis in the gut/pancreas and can thus be used to treat autoimmune diseases characterized by inflammation of the gut.

As used herein, a subject typically includes a mammal, more specifically a human subject. In one aspect, the subject is suffering from one or more symptoms of gut inflammation such as diarrhea, fever, fatigue, abdominal pain, abdominal cramping, blood in the stool, reduced appetite, and/or unintended weight loss. Administration of a cannabinoid receptor ligand can improve one or more of the symptoms of gut inflammation.

Also as used herein, the term gut refers to the gastrointestinal tract as well as liver, spleen, pancreas, omentum, and other organs served by the blood supply to and from the gut.

As used herein, a cannabinoid receptor ligand is a molecule that binds and stimulates or inhibits the cannabinoid receptor. While VR1 and CB2 were known to be expressed in neurons and dendritic cells, the inventors unexpectedly found that VR1 and CB2 are also expressed in the small intestinal propria. The cannabinoid receptor ligand that are useful in the methods disclosed herein include those previously identified as agonists of VR1.

Cannabinoids include Phytocannabinoids, Endocannabinoids, synthetic cannabinoids, and combinations thereof. Phytocannabinoids are cannabinoids that originate from nature and can be found in the cannabis plant. Endocannabinoids are the cannabinoids that are produced endogenously by human or animal bodies. Synthetic cannabinoids are compounds that have a cannabinoid-like structure yet are manufactured using chemical means. Depending on the method of manufacture the synthetic cannabinoid may comprise a racemic mixture of cannabinoids, in contrast to an isolated cannabinoid which will typically be a single enantiomer.

Phytocannabinoids include Tetrahydrocannabinols (such as, e.g., Delta-9-tetrahydrocannabinol (Delta-9-THC), and Delta-8-tetrahydrocannabinol (Delta-8-THC)), Cannabidiols, Cannabinols, Cannabigerols, Tetrahydrocannabivarins, Cannabidivarins, and Cannabichromenes.

Endocannabinoids include Arachidonoylethanolamine (Anandamide or AEA), 2-arachidonoyl glycerol (2-AG), 2-arachidonyl glyceryl ether (noladin ether), N-arachidonoyl-dopamine (NADA), Virodhamine (OAE), and Lysophosphatidylinositol (LPI).

Synthetic cannabinoids include Dronabinol (Marinol), Nabilone (Cesamet), Sativex, Rimonabant (SR141716), JWH-018, JWH-073, CP-55940, Dimethylheptylpyran, HU-210, HU-331, SR144528, WIN 55, 212-2, JWH-133, Levonantradol (Nantrodolum), and AM-2201.

In one embodiment, the method further comprises administering a second compound that is a vanilloid receptor 1 (VR1) agonist.

VR1 agonists include resiniferatoxin and other resiniferatoxin-like complex polycyclic compounds such as tinyatoxin, capsaicin and other capsaicin analogs such as ovanil, and other compounds that include a vanilloid moiety that mediates binding and activation of VR1. Additional VR1 agonists include capsiate, civamide, SDZ-249-665, DA-5016, arvanil, scutigeral, isovelleral, phorbol 12,13-didecanoate 20 homovanillate, phorbol 12,13-dinonanoate 20 homovanillate, and comparable substances, as well as analogs, derivatives and salts of the compounds mentioned above.

In certain embodiments, the cannabinoid receptor ligand is combined with a second active agent that is an FAAH inhibitor, an inhibitor of anandamide transporter, an inhibitor of anandamide amidase, or a combination thereof.

FAAH inhibitors are described in U.S. Pat. Nos. 8,293,724 and 8,202,893, included herein by reference for their disclosure of FAAH inhibitors. FAAH inhibitors can have the formula:

Ar—R-E,

wherein Ar is an optionally substituted aryl or heteroaryl, R is an aliphatic linker, and E is a moiety selected from trifluoromethyl ketone, boronic acid, boronic acid ester, fluorosulfone, fluorophosphonate, α-haloketone and α-haloester.

FAAH inhibitors also include N-(4-hydroxyphenyl)-arachidonylamide, palmitylsulphonylfluoride, arachidonyltrifluoromethylketone and 4-benzyloxyphenyl-n-butylcarbamate.

Inhibitors of anandamide transporters are disclosed in U.S. Pat. Nos. 7,589,220 and 8,202,893, incorporated herein by reference for their disclosure of inhibitors of anandamide transporters. Such inhibitors include compounds of the formula

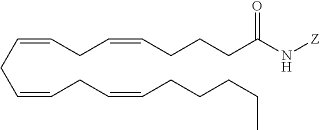

wherein Z is selected from a heterocyclic ring, a substituted heterocyclic ring, a heteroaromatic ring, or a substituted heteroaromatic ring.

Additional anandamide transporter inhibitors include AM404 and OMDM-1.

Inhibitors of anandamide amidase are described in U.S. Pat. No. 6,579,900, included herein by reference in its entirety for its disclosure of inhibitors of anandamide amidase. Additional anandamide amidase inhibitors include methyl arachidonyl fluorophosphonate.

Additional active agents that may be combined with the cannabinoid receptor ligands include standard-of-care drugs that are used to treat gut inflammation, such as 5-aminosalicylic acid, TNF inhibitors, azathioprine, methotrexate, 6-mercaptopurine, steroids or probiotics, and the like.

As used herein, an effective amount of a cannabinoid receptor ligand is an amount sufficient to reduce gut inflammation in a subject and/or to reduce the symptoms associated with gut inflammation in the subject such as diarrhea, fever, fatigue, abdominal pain, abdominal cramping, blood in the stool, reduced appetite, and/or unintended weight loss. Exemplary dosage amounts of the cannabinoid receptor ligand are 5 mg/kg body weight to 100 mg/kg body weight.

As used herein, “pharmaceutical composition” means therapeutically effective amounts of the cannabinoid receptor ligand with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

The active ingredient may be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anaesthetic, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

“Pharmaceutically acceptable salts” includes derivatives of the disclosed compounds wherein the parent compound is modified by making an acid or base salt thereof, and further refers to pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable.

The cannabinoid receptor ligands are used to improve the immune homeostasis in the gut of a subject suffering from an autoimmune disease characterized by inflammation of the gut. Many autoimmune diseases primarily affect the gastrointestinal tract, while other autoimmune diseases, including systemic autoimmune diseases such as lupus, result in gastrointestinal inflammation in addition to joint inflammation, for example. Exemplary autoimmune diseases that can be treated with the cannabinoid receptor ligands include autoimmune diseases primarily affecting the gastrointestinal tract such as celiac disease, Crohn's disease, ulcerative colitis, autoimmune hepatitis, inflammatory bowel disease, pernicious anemia, and primary biliary cirrhosis. Endocrinologic autoimmune diseases that have gut inflammation as a symptom include diabetes mellitus Type I, Hashimoto's thyroiditis, Grave's disease, and Addison's disease. Systemic autoimmune disorders that have gut inflammation as a symptom include systemic lupus erythematosus, Sjogren's syndrome, scleroderma, rheumatoid arthritis, and the like. While the treatment of autoimmune disease typically depends on the disease, a major goal of treatment is the reduction of inflammation. Thus, treatments that reduce gut inflammation would be helpful in treating a wide variety of autoimmune disorders.

The invention is further illustrated by the following non-limiting examples.

### EXAMPLES

### Methods

Mice: C57BL/6, NOD/Lt, VR1^{-/-} (also known as TRPV1^{-/-}), IL27RA^{-/-} (also known as WSX-1^{-/-}) and CX3CR1^{gfp/gfp}, were obtained from the Jackson Laboratory (Bar Harbor, Me.). IL10-GFP reporter (Vert-X) mice were a kind gift from Dr. Roger S Thrall. All animals except NOD/Lt were on a C57BL/6 background. CX3CR1^{gfp/gfp} were crossed with C57BL/6 mice to obtain CX3CR1^{gfp/+} mice. CX3CR1^{gfp/gfp} mice were backcrossed with VR1^{-/-} to obtain CX3CR1^{gfp/+} VR1^{-/-} mice. IL27RA^{-/-} mice were backcrossed with C57BL/6 mice to obtain IL27RA^{+/-} mice. All animals were housed in the Center for Comparative Medicine at UConn Health. All experiments were conducted under the guidelines approved by the UConn Health Animal Care Committee.

Cell isolation: The siLP cells were isolated as per protocols known in the art. In brief, small intestines were removed from the mice, luminal contents were flushed with cold HBSS (Invitrogen, Carlsbad, Calif., USA) with 5% FBS, opened longitudinally after removing Peyer's patches and cut in to small pieces. The epithelial cells were removed by incubation with HBSS containing 1.3 mM EDTA (Invitrogen, Carlsbad, Calif., USA), 1mM HEPES (Invitrogen, Carlsbad, Calif., USA) and 0.77 mM DTT (Sigma, St. Louis, Mo., USA) at 37° C. shaking at 350 rpm. The intestinal pieces were then washed with PBS (Invitrogen, Carlsbad, Calif., USA) and digested in 100 units/ml collagenase IV (Sigma, St. Louis, Mo., USA) at 37° C. shaking at 550 rpm. Leukocytes were enriched using percoll (GE Healthcare, Piscataway, N.J., USA) gradient. The PLN, MLN and splenic cells were isolated by gently crushing the organs through 70 μm cell strainer (BD Falcon, Franklin, N.J., USA). Splenocytes were incubated with ACK lysing buffer (Invitrogen, N.Y., USA) to eliminate red blood cells for 3 mins at 37° C. and washed twice with PBS.

Flow cytometry: Fluorescently labeled antibodies used for surface staining included: CD11b (M1/70), CD11 c (N418), CX3CR1 (polyclonal), CD103 (2E7), CD64 (X54-5/7.1), MHCII (M5/114.15.2), B220 (RA3-6B2), CD3 (145-2C11), CD4 (RM4-5), CD25 (PC61) and CD62L (ME2-14) and those used for intracellular staining include: VR1 (polyclonal) IL10 (JESS-16E3), TNFα (MP6-XT22), IFNγ (XMG1.2), and IL17A (TC11-18H10.1). For detection of CB2, primary antibody against CB2 (polyclonal) was used followed by fluorescently labeled secondary antibody. All were purchased from Biolegend San Diego, Calif., USA except for TNFα (Becton Dickinson, San Jose, Calif., USA), CX3CR1 (R&D Minneapolis, Minn., USA), VR1 (Thermo Scientific, Rockford, Ill. USA), IL10 (eBioscience, San Diego Calif., USA) and CB2 (Thermo Scientific, Rockford, Ill. USA). Cells were acquired with a LSRII flow cytometer (Becton Dickinson, San Jose, Calif., USA) or sorted with a FACSAria™ machine (Becton Dickinson, San Jose, Calif., USA). Flow cytometry analysis was done with the FlowJo Version 9.7.6 (Tree Star, Ashland, Oreg,).

Intracellular staining for VR1: After surface staining and fixing, cells were incubated in permeabilization buffer (BD Biosciences, San Jose, USA). Cells were then incubated in 4% goat serum for 30 minutes to prevent non-specific binding of the secondary antibody. After washing, cells were incubated with primary antibody against VR1 (rabbit anti-mouse) or unconjugated isotype control for 30 minutes. Next the cells were washed two times and stained with fluorochrome labeled anti-rabbit secondary antibody for 30 minutes. After washing two times cells were analyzed by flow cytometry.

Intracellular cytokine staining: Cells were incubated with PMA (50 ng/ml) (Sigma Co., St. Luis, Mo., USA) plus ionomycin (1 μg/ml) (Sigma Co., St. Luis, Mo., USA) in the presence of golgi plug (BD Biosciences, San Jose, USA) for 5 hours at 37° C. followed by surface staining with fluorochrome conjugated antibodies and then intracellular staining of IL10, TNFα, IFNγ, and IL17A was performed by fixation and permeabilization according to manufacturer's protocol BD Cytofix/Cytoperm™ kit.

In vitro co-culture assays: For in vitro co-culture assays, siLP MNPs were sorted as described previously from C57BL/6 or CX3CR1^{gfp/+} mice. MNPs (10×10⁴) were cocultured for 4 days with splenic naïve CD25⁻ CD62L^{hi} CD4^{±}T cells sorted from untreated C57BL/6 or IL27Ra^{-/-} mice at 1:1 ratios in the presence of soluble αCD3 mAb (145-2C11) (0.5 μg/ml) in U-bottomed 96 well plates. The cultured cells were maintained in RPMI 1640 medium supplemented with 5% fetal bovine serum, 1 mM sodium pyruvate, 1 mM non-essential amino acids, 2 mM L-gluatamine, 50 units/ml streptomycin, and 50 μM Beta-mercaptoethanol, all purchased from Gibco/Invitrogen (Carlsbad, Calif., USA). After 4 days, the cultured cells were washed with PBS and surface stained with CD4 and intracellular staining was performed for IL10 and IFN-γ.

Oral administration of CP, AEA and PF3845: Mice were anesthetized with intraperitoneal injection of ketamine (2 mg/kg) and xylazine (0.1 mg/kg). Vehicle control (tween 80 (5%), ethanol (85%) in PBS) or CP (10 μg) (ENZO Life Sciences, Farmingdale, N.Y., USA) or AEA (250 μg, 400 μg or 500 μg) (Cayman Chemical Co., Ann Arbor, Mich., USA) or PF3845 (5 mg/kg) (Cayman Chemical Co., Ann Arbor, Mich., USA) were gavaged in 100 μl volume using an 18-gauge feeding needle (Popper & Sons, New Hyde Park, N.Y., USA).

qPCR: Total RNA was isolated from sorted siLP MNP using the RNeasy® Micro kit Oaten, Chatsworth, Calif., USA). cDNA was made from total RNA using iSCRIPT™ cDNA synthesis kit (Bio-Rad Laboratories, Hercules, Calif.). IL-27 gene expression was measured using IL-27 TactMan® Gene Expression Assay (Life technologies, Grand Island, N.Y., USA) and CFX96™ Real-Time PCR detection system (Bio-Rad Laboratories, Hercules, Calif., USA). Gene expression was normalized to GAPDH.

Adoptive transfer: Female NOD mice were orally gavaged with CP 10 μg or vehicle at 9 and 10 weeks of age as described earlier. 2 weeks later CD4⁺ T cells were sorted from the PLN and spleen of the treated mice. All recipient female NOD mice were sub-lethally irradiated. Four hours after irradiation the recipient mice received 10×10¹⁰ diabetogenic splenocytes. 7×10⁶ sorted CD4⁺ T cells were then transferred to naïve NOD female mice by retro-orbital injection at 11 weeks of age. Development of diabetes was monitored by measuring urine glucose using Diastix® reagent strips (Bayer, Elkhart, Ind., USA). Mice were considered diabetic after two consecutive readings of >1000 mg/dl of urine glucose.

Statistical analyses: Data were analyzed by Student's t-test (unpaired, one-tailed) using GraphPad Prism Version 6.0 (GraphPad Software, La Jolla, Calif., USA). P<0.05 was considered significant.

### Example 1

### Expression of VR1 and CB2 on Cell Populations in the Small Intestinal Lamina Propria

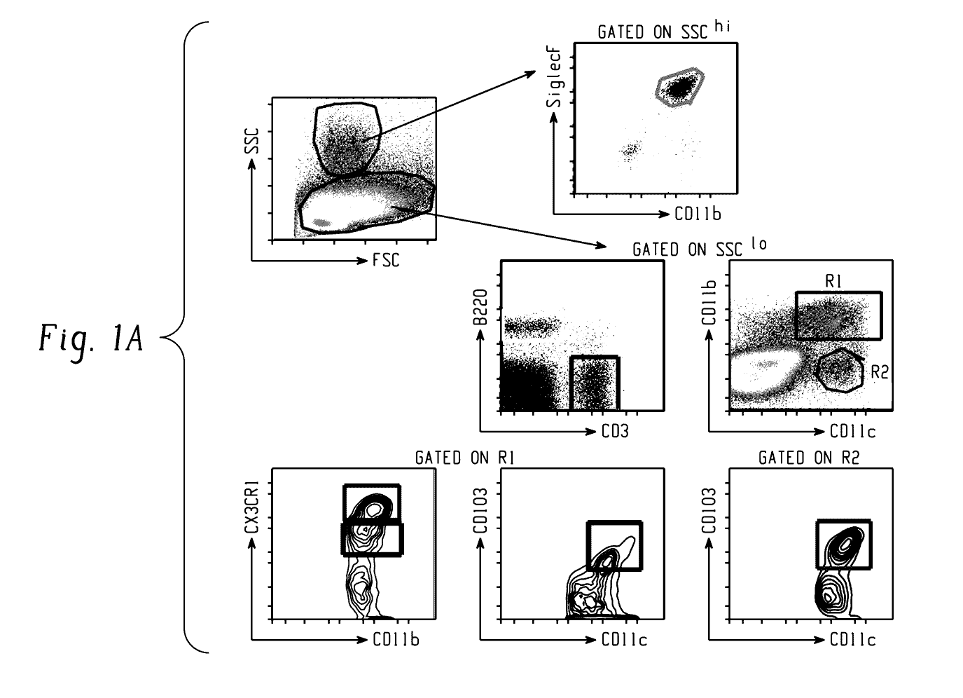

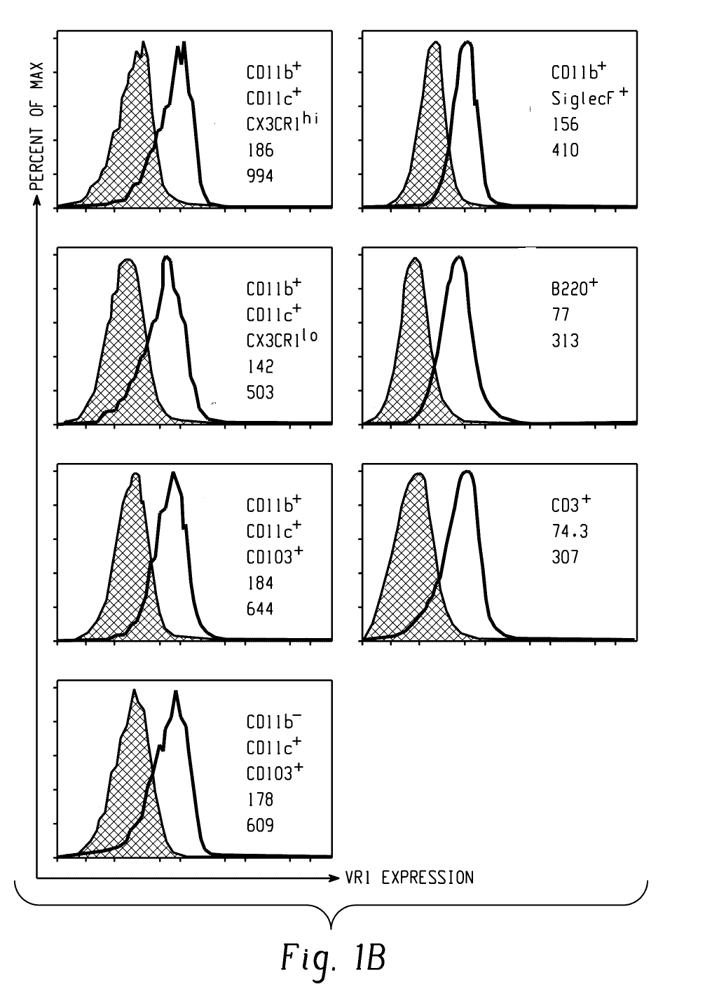

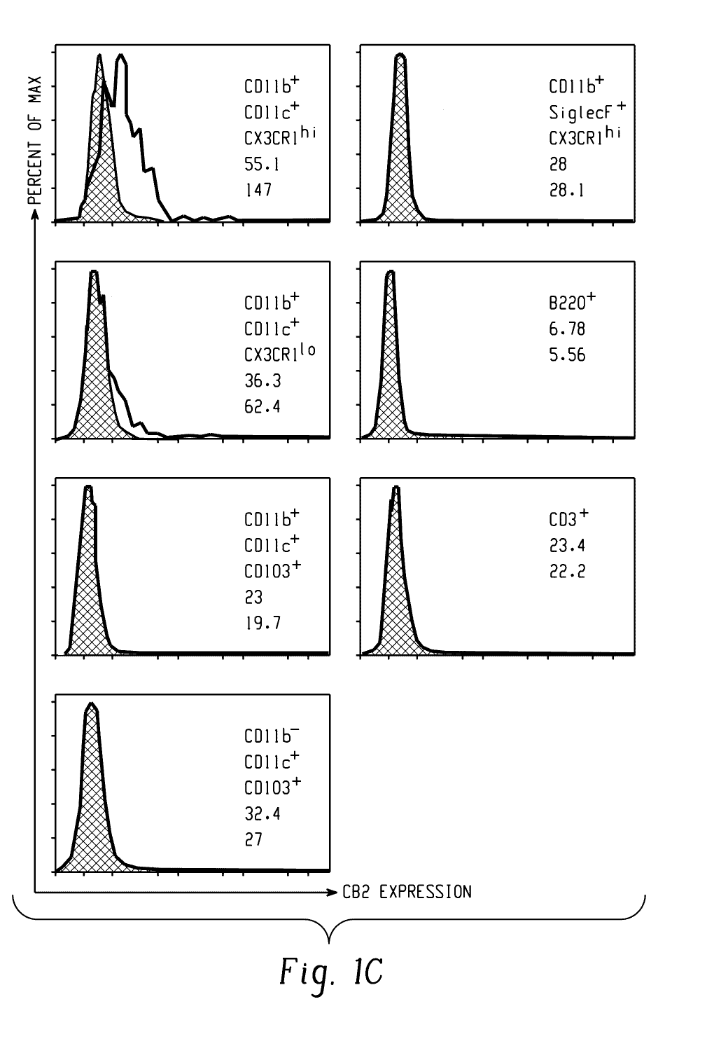

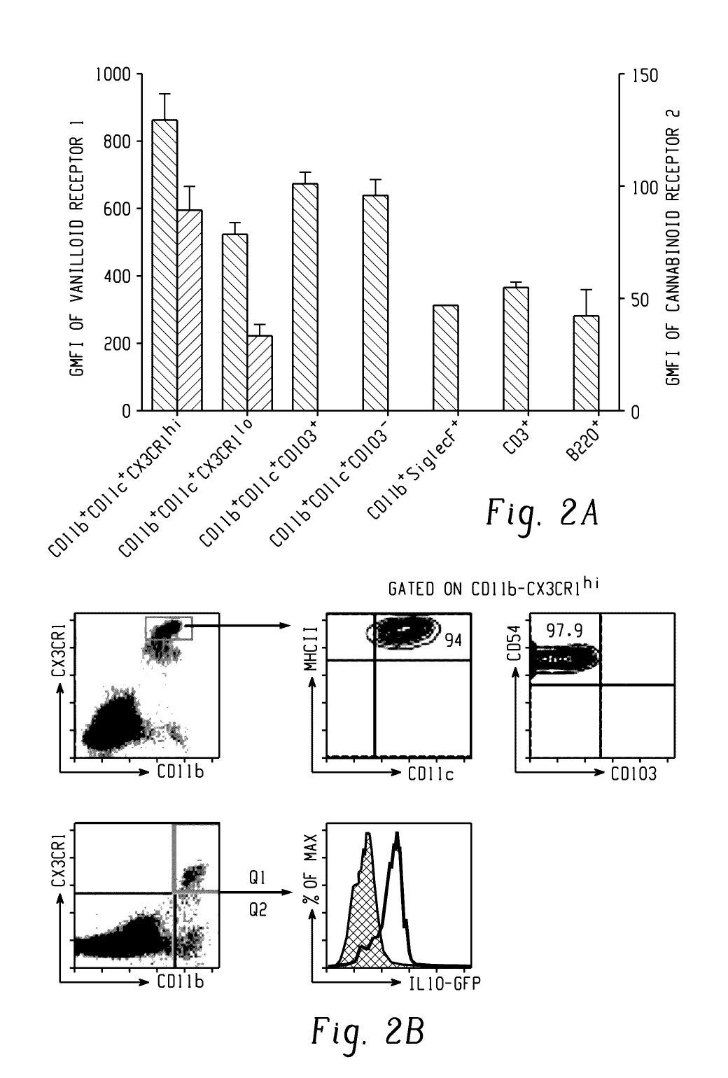

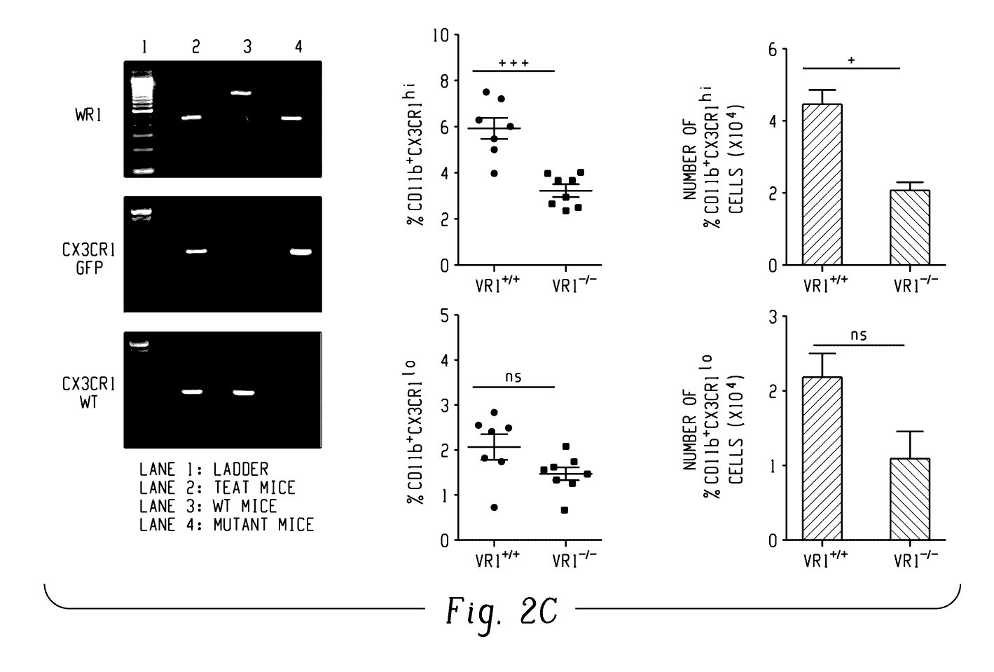

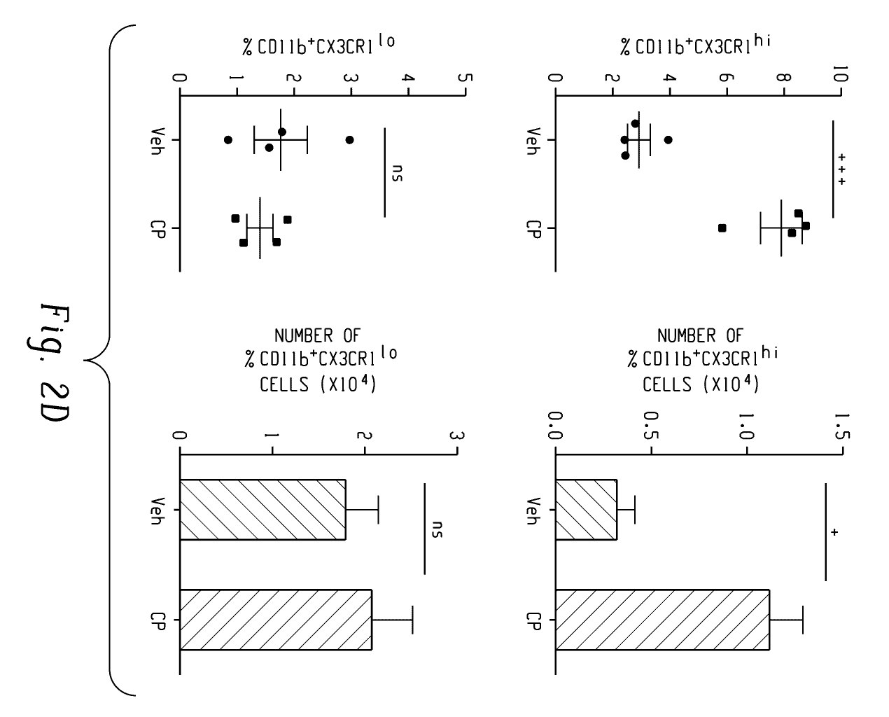

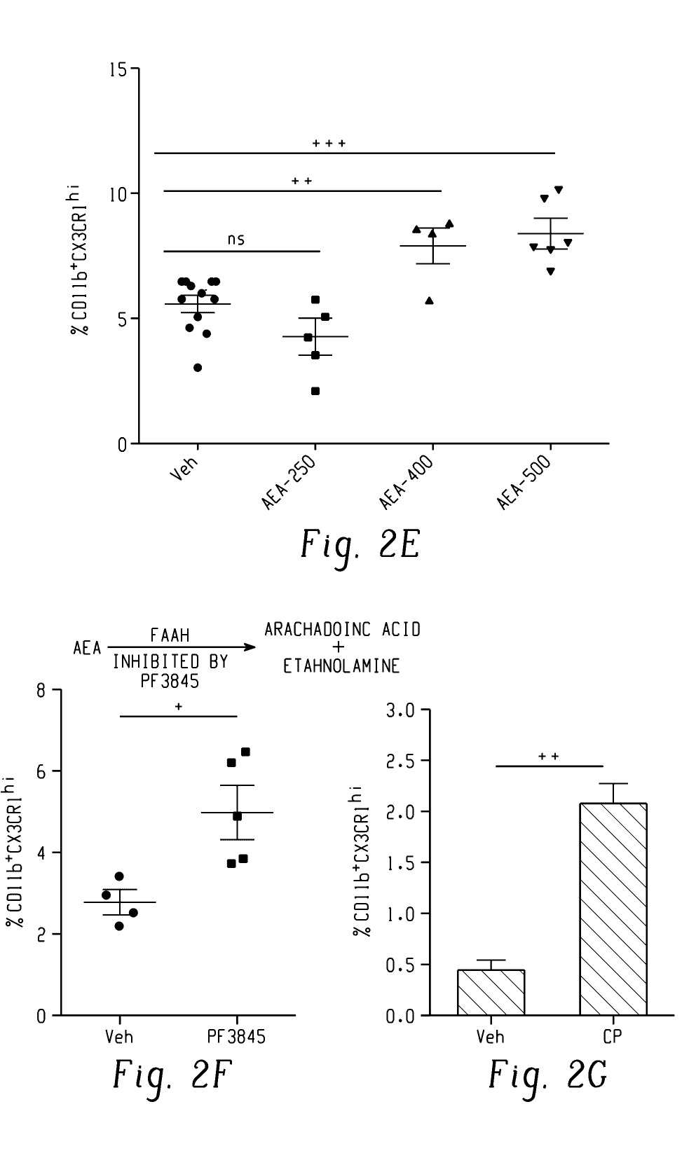

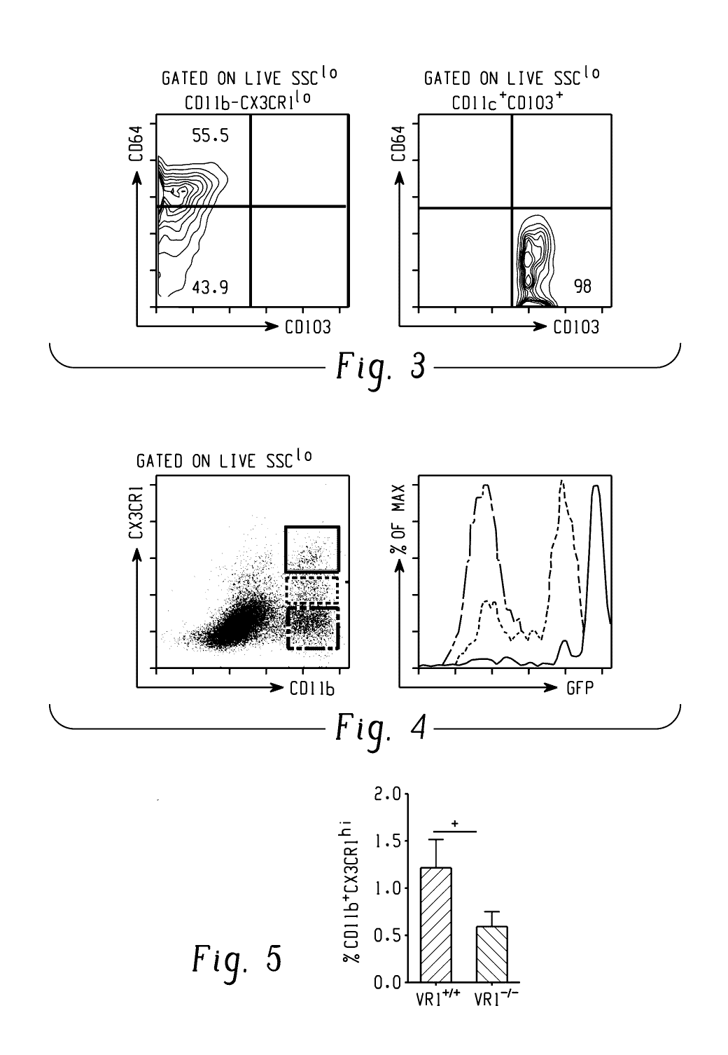

The expression of endocannabinoid receptors VR1 and CB2 on the major cell populations in the small intestinal lamina propria (siLP). See FIG. 1A for the gating strategy. In addition to the lymphocytes (B220+ and CD3+ ), characterization of the mononuclear phagocytes (MNPs) was based on the expression of integrins CD11b (αM), CD11c (αX), CD103 (αE) and the chemokine receptor CX3CR1. Special attention was paid to the CX3CR1 phenotype since CX3CR1ₕᵢ Mφ but not the CX3CR1ₗₒ cells play a significant role in immune homeostasis in the gut. To facilitate the identification of CX3CR1ₕᵢ and CX3CR1ₗₒ cells, we used the CX3CR1_{gfp/+} reporter mice where GFP expression is a marker for CX3CR1 expression. The MNPs expressed higher levels of VR1 and CB2 as compared to the lymphocytes and eosinophils. Strikingly, CX3CR1ₕᵢ cells expressed higher level of VR1 and CB2 as compared to all other cells (FIG. 2A, FIGS. 1B and 1C). CX3CR1ₕᵢ cells are bonafide regulatory Mφ that express high levels of CD64 and IL10 in addition to MHCII and CD11c (FIG. 2B) as described, whereas CX3CR1ₗₒ cells and CD103+ DCs do not meet these criteria (FIG. 3). The pattern of antibody staining of CX3CR1 is consistent with that of the GFP expression in CX3CR1_{gfp/+} reporter mice (FIG. 4).

VR1_{-/-} mice were back-crossed with CX3CR1_{gfp/gfp} reporter mice to obtain CX3CR1_{gfp/+} VR1_{-/-} mice (FIG. 2C), in which the CX3CR1111 MO can be monitored through GFP expression. The frequency and absolute numbers of CX3CR1ₕᵢ Mφ, but not those of CX3CR1ₗₒ cells are reduced significantly in these VR1_{-/-} mice (FIG. 2C). These results were also reproduced in VR1_{-/-} mice where CX3CR1 was identified by antibody staining (FIG. 5). Importantly, the frequency and numbers of the other cells such as CD103+ DCs, CD3+ and B220+ cells remain unaffected in the absence of VR1 (FIG. 6).

### Example 2

### Oral Gavage with CP and AEA

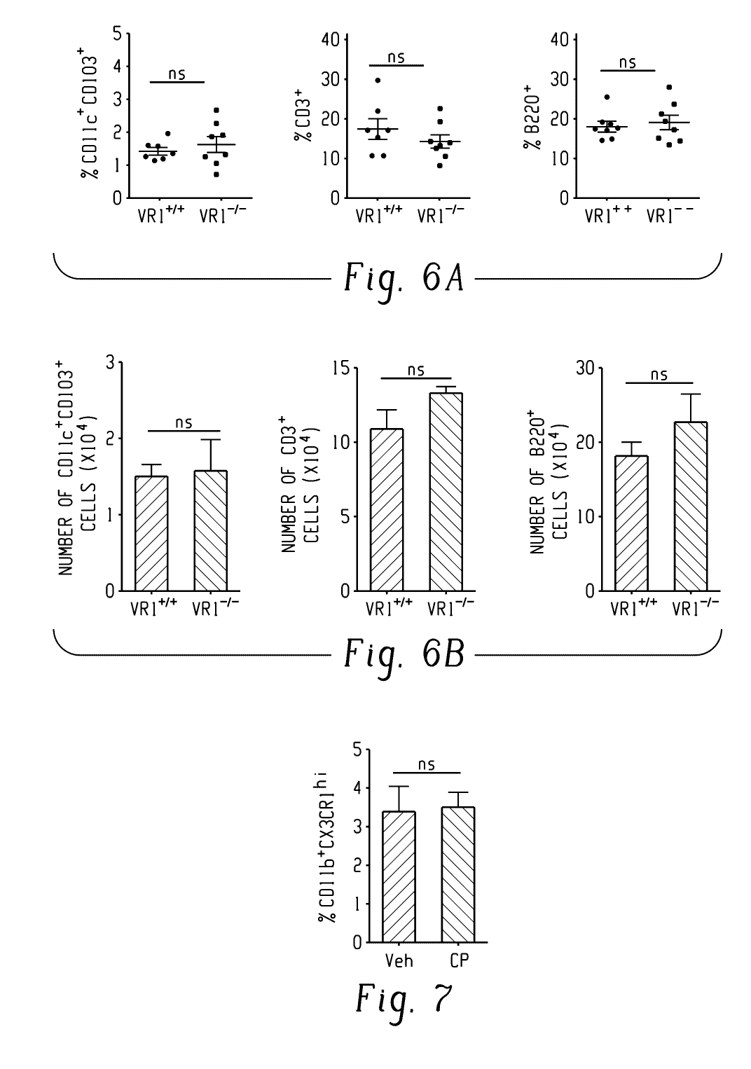

We orally gavaged CX3CR1_{gfp/+} mice with VR1 ligands CP or AEA. In case of CP (10 μg), its administration significantly increased the frequency of the regulatory CX3CR1ₕᵢ Mφ but did not affect the frequency and number of the CX3CR1ₗₒ cells (FIG. 2D). Interestingly, feeding CP to CX3CR1_{gfp/gfp} mice, i.e. mice which lack functional CX3CR1 protein, no changes in the frequency of the CX3CR1ₕᵢ Mφ in the siLP were noted, demonstrating that VR1 mediated homeostasis of these Mφ requires the presence of functional CX3CR1 (FIG. 7). Similar to the observations with CP, feeding mice with AEA led to a significant increase in the frequency of CX3CR1ₕᵢ Mφ (FIG. 2E). AEA is generated in the gut and its levels can be regulated by fatty acid amide hydrolase (FAAH), which catabolizes it (FIG. 2F) Inhibition of FAAH leads to increase in the levels of AEA. Treating mice with FAAH inhibitor PF3845 also led to an increase in the frequency of the CX3CR1ₕᵢ Mφ (FIG. 2F), similar to the observation made by feeding mice with AEA. CP administration also increases the frequency of CX3CR1ₕᵢ Mφ in NOD mice, a model of type 1 diabetes that we use later in this study (FIG. 2G).

### Example 3

### Effect of VR1 on the Tolerogenic Properties of siLP MNPs

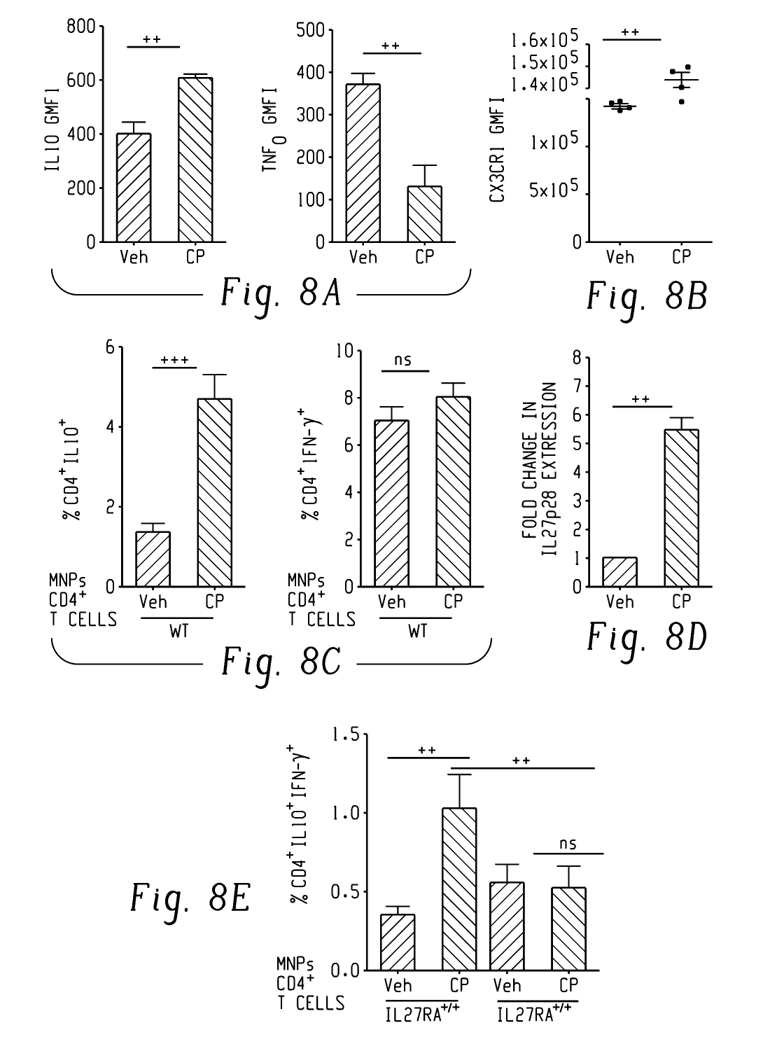

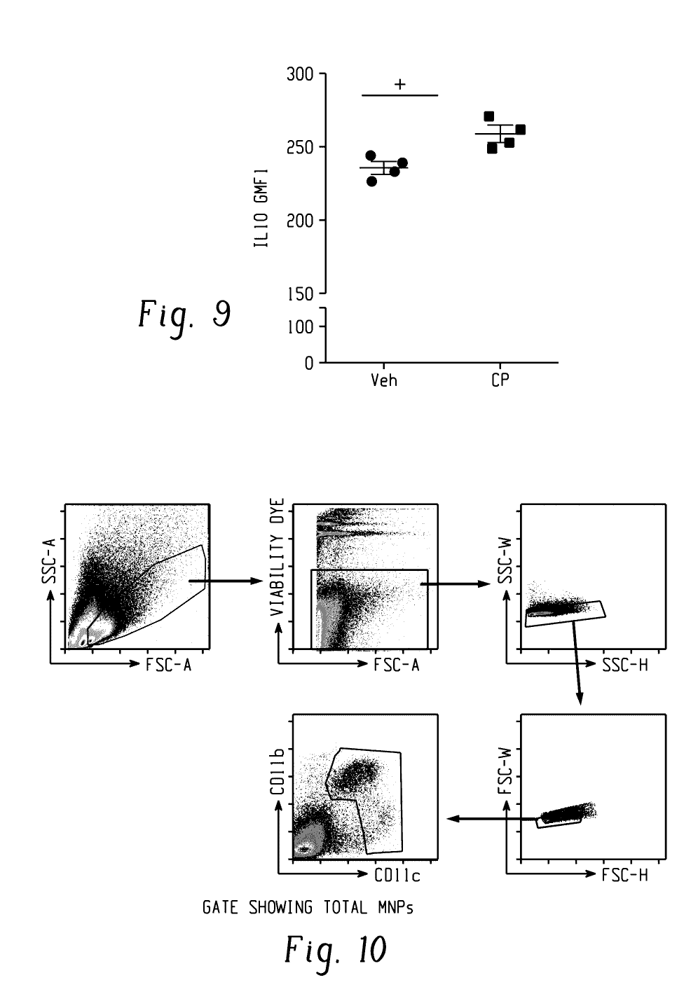

Since IL10 is a cytokine with potent tolerogenic properties in the gut, we looked at the IL10 production as a measure of tolerogenicity in the CX3CR1ₕᵢ Mφ and CD103₊ DCs 24 hours after treatment with CP using the IL10 GFP-reporter mice. Compared to controls, the CX3CR1ₕᵢ Mφ of CP fed mice showed a significant increase in the production of IL10 (FIG. 8A, left panel). In contrast the production of TNF-α, an pro-inflammatory cytokine, in the CX3CR1ₕᵢ Mφ was decreased (FIG. 8A, right panel). Additionally, CP treatment of CX3CR1_{gfp/+} mice led to a significantly increased expression of CX3CR1 on these Mφ (FIG. 8B). This was particularly important, as it has been shown earlier that the regulatory properties of CX3CR1ₕᵢ Mφ depend on the expression of functional CX3CR1 protein itself. CP treatment also resulted in a modest but statistically significant increase in IL10 production in CD103+ DCs (FIG. 9).

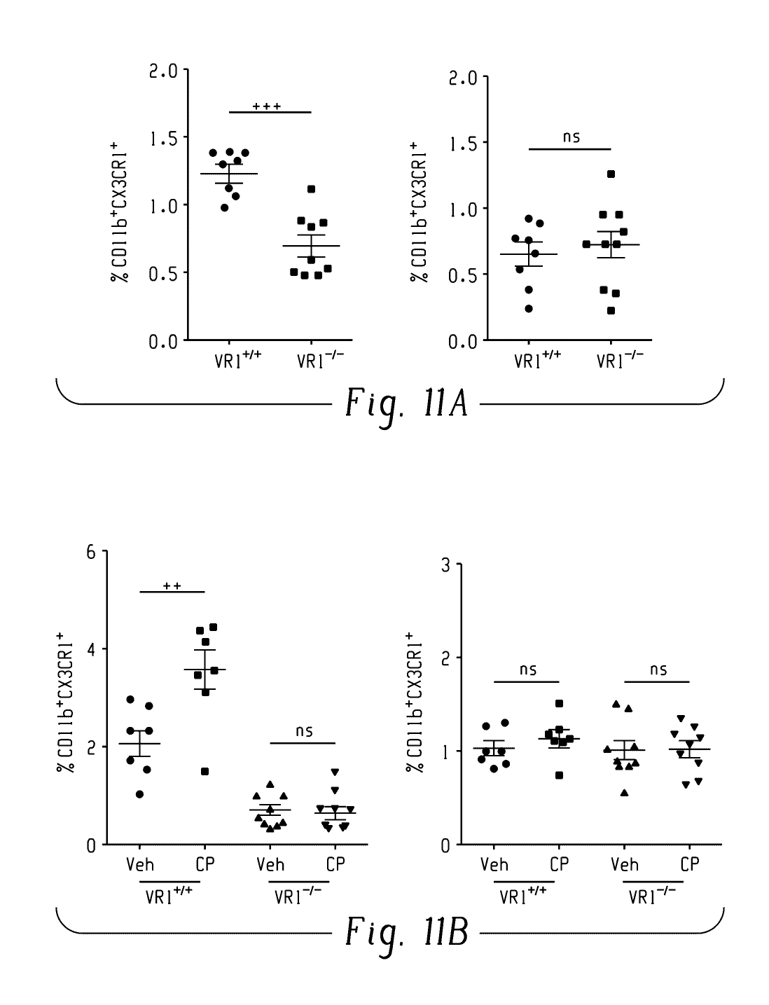

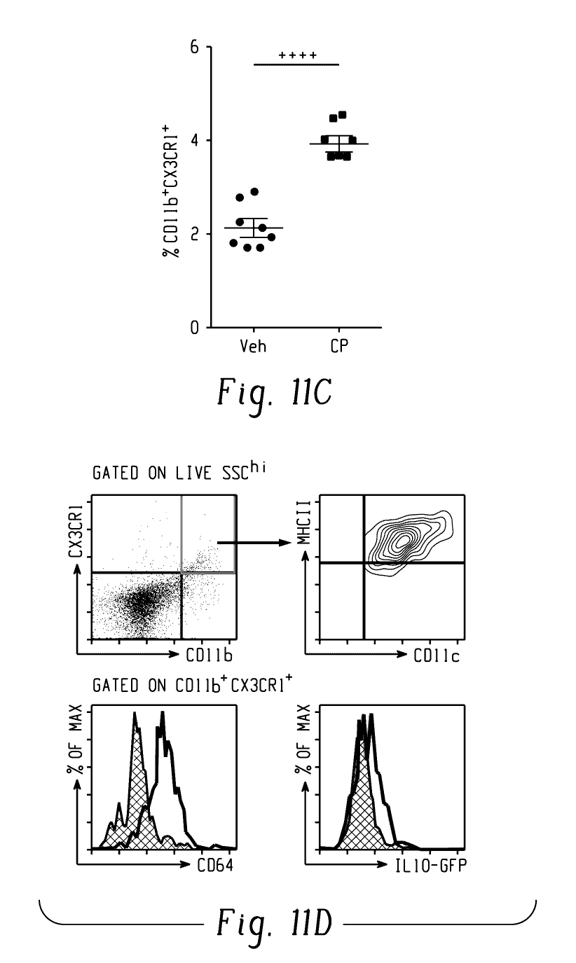

The ability of CP to alter the ability of the siLP MNPs to induce IL10-producing regulatory T cells was tested. The CX3CR1ₕᵢ Mφ and CD103₊ DCs constitute the overwhelming majority of siLP MNPs, thus the total MNPs (CD11b₊CD11c₊ and CD11b₋CD11c₊ cells) (FIG. 10) were sorted from mice treated with vehicle or CP 24 hours earlier and co-cultured with naïve splenic CD4₊ T cells (1:1) for four days, and IL10 and IFN-γ producing CD4₊ T cells were analyzed. CP elicited MNPs led to a significant increase in the expression of IL10 but not IFN-γ in the CD4₊ T cells (FIG. 8C, upper panel). Interestingly, CP elicited MNPs also led to significant increase in the induction of a population of CD4₊ T cells that express both IL10 and IFN-γ(FIG. 8C, lower panel). CD4₊ T cells that produce both IL10 and IFN-γ have been previously described as a potent regulatory subset called Tr1. IL27, a member of the IL12 family, plays a dominant role in the induction of Tr1 cells. The sorted MNPs were co-cultured with naïve CD4₊ T cells from IL27RA_{+/+} (WT) or IL27RA_{-/-}: the increase in the frequency of CD4₊ IL10₊ IFN-γ₊ T cells seen in CP-elicited MNPs is completely abrogated in the T cells derived from the IL27RA_{-/-} mice (FIG. 8C, lower panel). The possibility that CP treatment induces IL27 production in the MNPs was tested. IL27-p28 levels were quantified by qPCR: two days after CP treatment, there was a five fold increase in the level of IL27-p28 in the MNPs (FIG. 8D).

### Example 4

### Role of VR1 in the Homeostasis of the Pancreatic (PLN) and Mesenteric Lymph Nodes (MLN)

Both the pancreatic (PLN) and mesenteric lymph nodes (MLN) drain the gut. The possibility that the absence of VR1 in addition to affecting the number of CX3CR1ₕᵢ Mφ in the siLP also affects the frequency of CX3CR1₊ Mφ in the gut draining lymph nodes i.e., PLN and MLN was tested. VR1_{-/-} mice showed a significant reduction in the frequency of CX3CR1₊ Mφ in the PLN (FIG. 11A, left panel) but not in the MLN (FIG. 11A, right panel). A significant VR1-dependent increase in the frequency of CX3CR1₊ Mφ was also observed in the PLN but not in MLN, 3 days after feeding CP (FIG. 11B, PLN, left and MLN, right). Feeding CP to NOD mice also resulted in significant increase in the frequency of CX3CR1₊ Mφ in the PLN (FIG. 11C).

The phenotype of the PLN CX3CR1₊ Mφ was akin to that of the siLP Mφ (shown in FIG. 2B) in terms of expression of CX3CR1, CD11c, MHCII, CD64 and spontaneous production of IL10 (FIG. 8D); in addition, changes in the siLP Mφ were seen as early as 24 hours after feeding with CP, while they were only seen after 3 days in the PLN. The phenotypic similarity of the siLP and PLN Mφ and the kinetics of the changes in them suggest that these Mφ migrate from the gut to the PLN.

### Example 5

### Engagement of VR1 Expands a Tolerogenic Subset of CD4+T Cells in an IL27 Dependent Manner

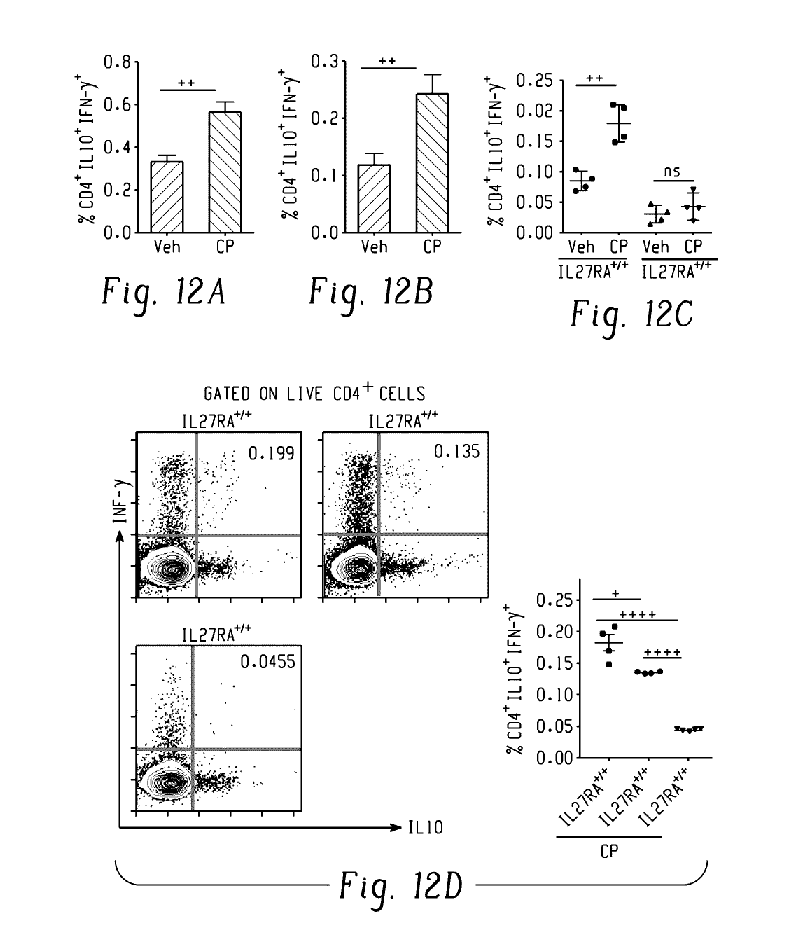

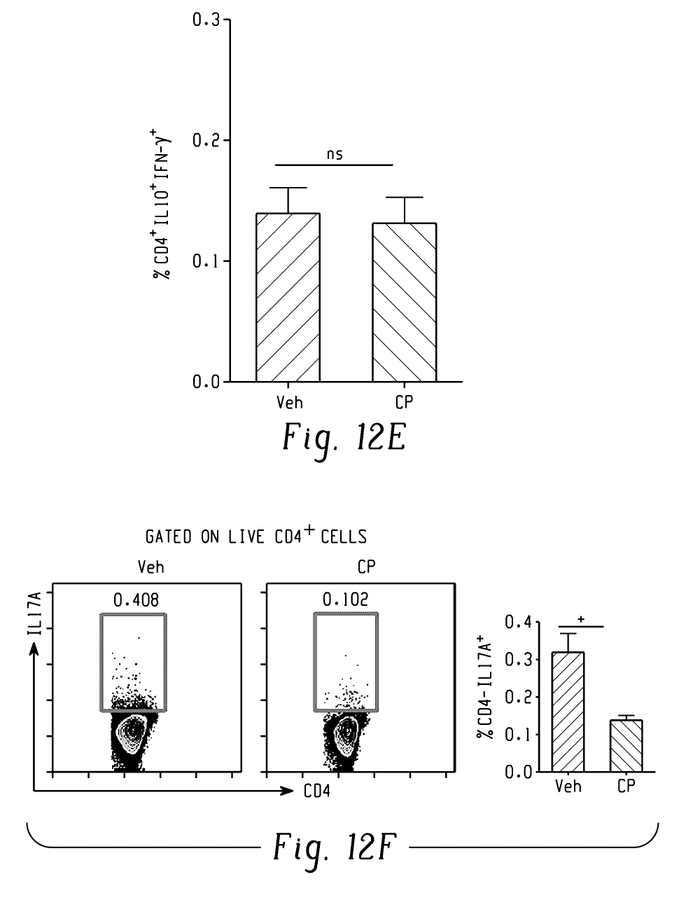

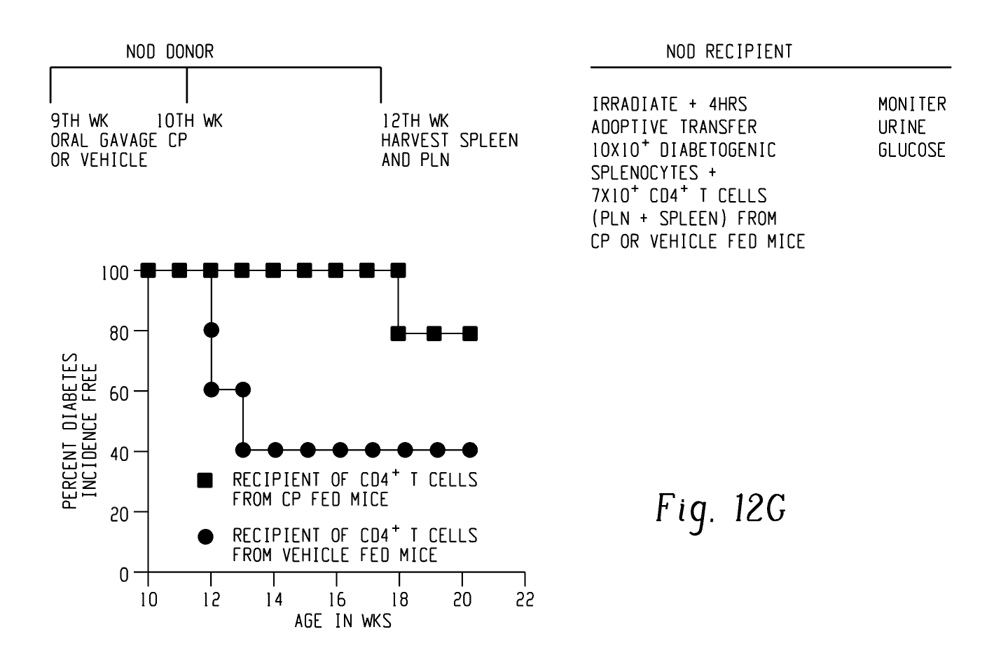

FIG. 8C showed that CP elicited MNPs mediate the differentiation of naïve CD4₊ T cells into Tr1 cells in vitro. We now tested if CP can elicit such regulatory T cells in vivo. NOD mice were orally gavaged twice at 9ₜₕ and 10ₜₕ week of age with 10 μg CP or vehicle as a control; two weeks post CP treatment, siLP and the PLN were harvested and the frequency of CD4₊IL10₊IFN-γ₊ Tr1 cells was measured. Consistent with the differentiation of Tr1 cells in vitro (FIG. 8C), CP-fed mice showed expansion of Tr1 cells in siLP (FIG. 12A) and PLN (FIG. 12B). Consistent with our study in vitro, CP-elicited Tr1 cells require the presence of functional IL27-IL27R signaling (FIG. 12C). Surprisingly, when the effects of IL27 signaling were tested in CP-fed mice that were IL27RA (−/−), hemizygous (+/−) and homozygous (+/+), a gene dose-dependence of Tr1 cell elicitation was observed (FIG. 12D). Such dose-dependency of IL27 signaling is a novel observation suggesting that the quantity of IL27RA is the limiting factor in physiological conditions.

Given that the CX3CR1ₕᵢ Mφ are critical to VR1 induced tolerance we asked if functional CX3CR1 is required for CP-elicited expansion of Tr1 cells. CX3CR1_{gfp/gfp} mice were orally gavaged with CP or vehicle as a control and Tr1 cells were analyzed in the PLN and it was observed that in the absence of functional CX3CR1 protein, CP does not elicit Tr1 cells (FIG. 12E). Of note, we found that oral administration of CP also leads to a significant decrease in the frequency of IL17A+ CD4+ T cells in the PLN (FIG. 12F). The functional consequence of this observation was tested in NOD mice; CD4+ T cells were isolated from the PLN and spleen of CP or vehicle treated female NOD mice. Naive female NOD mice received the CD4+ T cells that were derived either from the CP or vehicle treated NOD donors. Diabetes was monitored by the urine glucose levels. CP elicited CD4+ T cells provided significantly higher protection from T1D as compared to the vehicle elicited CD4+ T cells (FIG. 12G).

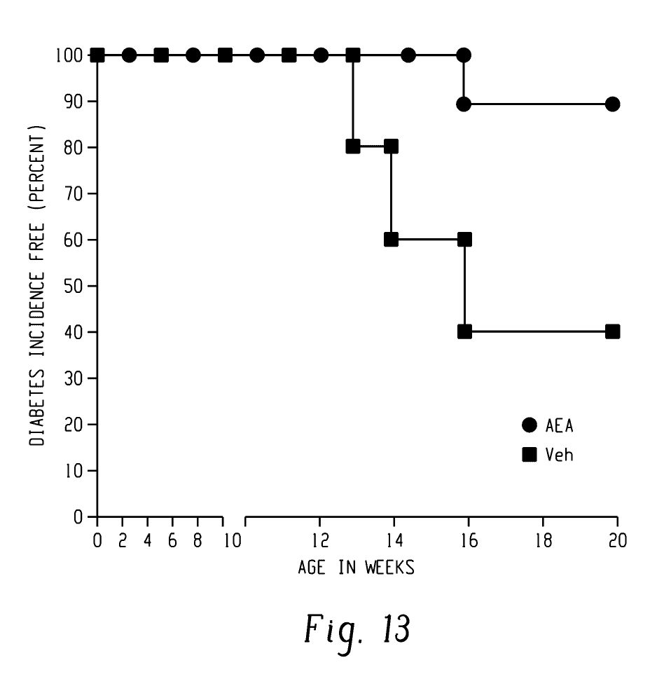

FIG. 13 further shows that oral administration of Anandamide (AEA) provides protection from Type 1 Diabetes (T1D). Female NOD mice were orally administered AEA or vehicle at 9^{th} and 10^{th} week of age and urine glucose was monitored to study T1D disease progression (n=10, *p<0.05, Mantel-Cox test).

These results establish the endocannabinoid system as a major participant in maintaining tolerance in the gut. It does so through two distinct, non-overlapping and complementary, mutually re-enforcing pathways, i.e. through its profound role in maintenance/differentiation of well-known immune regulatory CX3CR1ₕᵢ Mφ population, and by mediating expansion of the regulatory Tr1 T cells. Remarkably, although our enquiry began with examination of the role of VR1 in immune tolerance elicited by CP, the most established exogenous ligand of VR1, the endocannabinoid AEA, also causes similar consequences as CP. This is the first example of a neurologically active endocannabinoid playing a substantial immunological role, and suggests interesting possibilities of concordant regulation of immune tolerance and energy balance through one ligand and two receptors.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of improving immune homeostasis in the gut of a subject suffering from an autoimmune disease characterized by inflammation of the gut, comprising
administering to the subject an effective amount of a cannabinoid receptor ligand to improve immune homeostasis in the gut of the subject, wherein the gut includes the gastrointestinal tract as well as organs served by the blood supply to and from the gut.

2. The method of claim 1, wherein administering is orally administering.

3. The method of claim 1, wherein the subject is suffering from one or more symptoms of gut inflammation and improving immune homeostasis in the gut improves one or more of the symptoms.

4. The method of claim 3, wherein the symptom is diarrhea, fever, fatigue, abdominal pain, abdominal cramping, blood in the stool, reduced appetite, unintended weight loss, or a combination thereof.

5. The method of claim 1, wherein the cannabinoid receptor ligand is a Cannabinoid.

6. The method of claim 5, wherein the Cannabinoid is an Endocannabinoid, a Phytocannabinoid, a synthetic cannabinoid, or a combination thereof.

7. The method of claim 6, wherein the Endocannabinoid is Anandamide, 2-arachidonoyl glycerol, 2-arachidonyl glyceryl ether, N-arachidonoyl-dopamine, Virodhamine, or Lysophosphatidylinositol.

8. The method of claim 1, further comprising administering a second active agent, wherein the second active agent is a vanilloid receptor 1 agonist, an FAAH inhibitor, an inhibitor of anandamide transporter, an inhibitor of anandamide amidase, or a combination thereof.

9. The method of claim 8, wherein the vanilloid receptor 1 agonist is resiniferatoxin, tinyatoxin, capsaicin, or ovanil.

10. The method of claim 8, wherein the second active agent is N-(4-hydroxyphenyl)-arachidonylamide, palmitylsulphonylfluoride, arachidonyltrifluoromethylketone, 4-benzyloxyphenyl-n-butylcarbamate, AM404, OMDM-1, or methyl arachidonyl fluorophosphonate.

11. The method of claim 1, further comprising administering a second agent which is a standard of care agent for the treatment of gut inflammation.

12. The method of claim 11, wherein the second agent is 5-aminosalicylic acid, TNF inhibitors, azathioprine, methotrexate, 6-mercaptopurine, a steroid, or a probiotic.

13. The method of claim 1, wherein the effective amount of the cannabinoid receptor ligand is 5 mg/kg body weight to 100 mg/kg body weight.

14. The method of claim 1, wherein the subject has a gastrointestinal autoimmune disease, an endocrinologic autoimmune disease, or a systemic autoimmune disease.

15. The method of claim 14, wherein the gastrointestinal autoimmune disease is celiac disease, Crohn's disease, ulcerative colitis, autoimmune hepatitis, inflammatory bowel disease, pernicious anemia, or primary biliary cirrhosis.

16. The method of claim 14, wherein the endocrinologic autoimmune disease is diabetes mellitus Type I, Hashimoto's thyroiditis, Grave's disease, or Addison's disease.

17. The method of claim 14, wherein the systemic autoimmune disease is systemic lupus erythematosus, Sjogren's syndrome, scleroderma, or rheumatoid arthritis.

18. A method of improving the symptoms of gut inflammation associated with diabetes mellitus Type I in a subject suffering from diabetes mellitus Type I, comprising administering to the subject an effective amount of a cannabinoid receptor ligand to reduce the symptoms of gut inflammation in the subject.

19. The method of claim 18, wherein administering is orally administering.

20. The method of claim 18, wherein the cannabinoid receptor ligand is an Endocannabinoid, a Phytocannabinoid, a synthetic cannabinoid, or a combination thereof.
